(19) United States
(12) Patent Application Publication
(10) Pub. No.: US 2016/0331592 A1
(43) Pub. Date: Nov. 17, 2016

(54) INTERACTIVE HELMET WITH DISPLAY OF WELDING PARAMETERS

(71) Applicant: Lincoln Global, Inc., City of Industry, CA (US)

(72) Inventor: Eric STEWART, Girard, PA (US)

(21) Appl. No.: 14/708,860

(22) Filed: May 11, 2015

(51) Int. Cl. A61F 9/06; B23K 9/00; B23K 37/00; B23K 9/095

(52) U.S. Cl. CPC A61F 9/06; B23K 9/095; B23K 9/00; B23K 37/006

(57) ABSTRACT

A welding system includes welding equipment for generating a welding current and voltage, a welding control system for controlling the welding current and voltage, a monitoring system for monitoring a welding parameter, and a welding helmet. The welding helmet includes a main body, a retinal tracking system configured to identify a field of view of a user by tracking movement of the user's eyes, and a visual display system configured to generate a visual image of the welding parameter and further configured to position the visual image within the field of view of the user identified by the retinal tracking system. The retinal tracking system is further configured to receive a welding parameter input from the user based on tracking movement of the user's eyes, and to communicate the welding parameter input to the welding control system to control at least one of the welding current and voltage.

### TECHNICAL FIELD

This invention relates in general to equipment used in welding, and more particularly to a welding system including an interactive welding helmet.

### BACKGROUND

Welding is an important process in the manufacture and construction of various products and structures. Applications for welding are widespread and used throughout the world, for example, the construction and repair of ships, buildings, bridges, vehicles, and pipe lines, to name a few. Welding may performed in a variety of locations, such as in a factory with a fixed welding operation or on site with a portable welder.

In manual or semi-automated welding a user/operator (i.e. welder) directs welding equipment to make a weld. For example, in arc welding the welder may manually position a welding rod or welding wire and produce a heat generating arc at a weld location. In this type of welding the spacing of the electrode from the weld location is related to the arc produced and to the achievement of optimum melting/fusing of the base and welding rod or wire metals. The quality of such a weld is often directly dependent upon the skill of the welder.

Welders generally rely upon a variety of information when welding. This information includes, for example, current and voltage and wire feed speed. Traditionally, welders would need to look at gauges on the control panel of the welding equipment to gain this information. This would require the welder to direct their field of vision away from the welding work area and as such was undesirable.

In the past, efforts have been made to provide welders with information during welding, such as in the method disclosed in U.S. Pat. No. 4,677,277, where current and voltage are monitored to produce an audio indication to the operator as to the condition of the arc in arc welding. However, monitors consisting only of audio arc parameter indicators are hard to hear and interpolate and are not capable of achieving the desired closeness of control and quality of weld often required.

More recently, as disclosed in U.S. Pat. No. 6,242,711, an apparatus for monitoring arc welding has been developed that provides a welder with real-time voltage and current conditions of the welding arc where information in the form of lights, illuminated bar graphs, light projections, illuminated see-through displays, or the like are placed within the visual range of the helmet wearing operator and located in proximity to the helmet viewing window in the helmet. However, in this apparatus a welder must still move their visual focus away from the welding work area in order to focus on the information located proximate to the welding window or the welder must accept the information peripherally while continuing to focus on the welding work area.

### SUMMARY

This invention relates to an interactive welding helmet that is capable of both displaying and receiving modifications of welding parameters in a hands-free manner using one or more of eye-control and/or voice control. The disclosed interactive welding helmet is also adapted for use with remotes, including both wired and wireless remotes, digital welding guns, TIG torches, and other equipment for welding or related operations.

Presently disclosed is a welding system. In various embodiments, the welding system includes welding equipment for generating a welding current and voltage, a welding control system for controlling the welding current and voltage, a monitoring system for monitoring a welding parameter, and a welding helmet. The welding control system may also control a wire feeder at a desired wire feed speed for the welding operation. The welding helmet includes a main body, a retinal tracking system configured to identify a field of view of a user by tracking movement of the user's eyes, and a visual display system configured to generate a visual image of the welding parameter and further configured to position the visual image within the field of view of the user identified by the retinal tracking system. In some embodiments, the visual image includes alpha-numeric characters. In some embodiments, the welding helmet further includes a microphone and is configured to respond to one or both of eye-control or voice-control as discussed below. In some embodiments, the welding parameter includes at least one or more of the welding current, the welding voltage, and a wire feed speed

In some embodiments, the retinal tracking system is further configured to receive a welding parameter input from the user based on tracking movement of the user's eyes, and to communicate the welding parameter input to the welding control system to control at least one of the welding current and voltage.

In some embodiments, the visual display system is further configured to display a menu of welding parameters, and the retinal tracking system is further configured to select a menu item from the menu of controllable welding parameters based on tracking movement of the user's eyes. In some embodiments, the visual display system is further configured to generate one or more status indicators each associated with a monitored welding parameter, and each status indicator indicates a relationship between the monitored welding parameter and an acceptable range for the monitored welding parameter defined by a welding procedure.

In some embodiments, the retinal tracking system is further configured to select a welding parameter, and the welding helmet further comprises a microphone configured to receive audible commands from the user to adjust the welding parameter to control at least one of the welding current and voltage. In some embodiments, the retinal tracking system includes a monocle positioned between one of a user's eyes and the visual display system.

In some embodiments, the visual display system is a window including a welding lens. In some embodiments, the visual display system is capable of projecting a stereogram on the welding lens. In some embodiments, the visual display system includes a series of mirrors for reflecting the visual image toward the welding lens. In some embodiments, the welding helmet includes a reflective surface proximate the welding lens for reflecting the visual image toward the interior of the welding helmet. In some embodiments, the visual display system comprises a holographic projection system. In some embodiments, the visual display system comprises includes an LCD display. In some embodiments, the visual display system comprises includes an LED array.

In some embodiments, the visual display system includes at least one video monitor for displaying a picture of an associated welding work area. In some embodiments, the welding helmet further includes at least one camera connected to the main body for providing the picture of the associated welding work area. In some embodiments, the visual display system is configured to include the visual image of the welding parameter in the picture of the associated welding work area.

In some embodiments, the focus of the visual image is at the same focal distance as the associated welding work area.

### BRIEF DESCRIPTION OF THE DRAWINGS

Reference is made to the accompanying drawings in which particular embodiments and further benefits of the invention are illustrated as described in more detail in the description below, in which:

FIG. 1 is a schematic view of a welding system according to the present invention.

FIG. 2 is an enlarged view of a welding helmet similar to the helmet of FIG. 1 including a camera.

FIG. 3 is a cross-sectional diagram of a welding helmet similar to the helmet of FIG. 2 including a projector.

FIG. 4 is a cross-sectional diagram of a welding helmet similar to the helmet of FIG. 3 including an integrated video display.

FIG. 5 is a perspective view of a welding helmet similar to the helmet of FIG. 2 including binocular cameras.

FIG. 6 is an interior view of a welding helmet similar to the helmet of FIG. 5 showing binocular viewing screens.

FIGS. 7A-B are an exemplary display/menu for use with the welding system according to the present invention.

### DETAILED DESCRIPTION

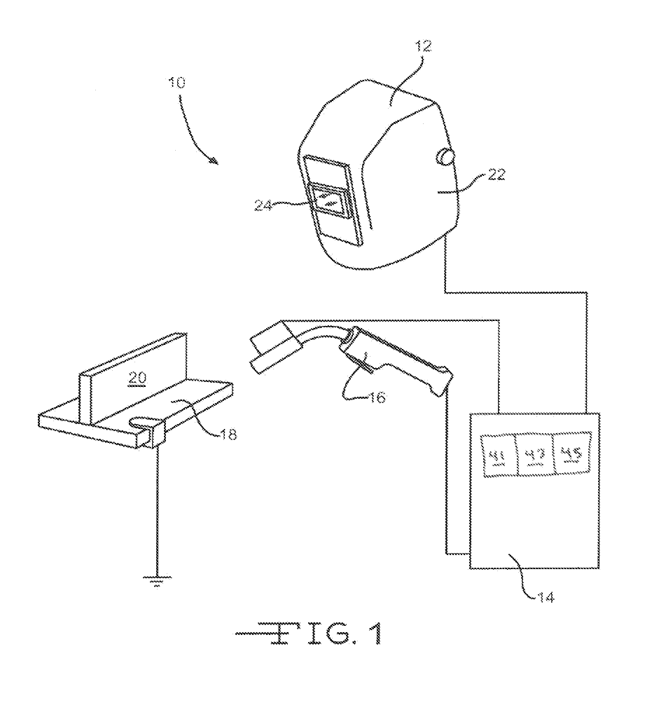

Referring generally to FIGS. 1-7, a welding system is disclosed that includes an interactive welding helmet. The interactive welding helmet is configured to both display welding parameters within the visual field of the welder. The interactive welding helmet may also enable the welder to adjust welding parameters in response to one or more of eye movements or voice commands, allowing the welder to modify the welding process while maintaining focus on the welding work area. As described below, the welding helmet may include a retinal tracking system that is capable of identifying a welder's visual field and receiving commands to modify welding parameters based upon tracking the movement of the welder's eyes. In some embodiments, the welding helmet also includes a microphone.

Referring now FIG. 1, a welding system 10 is illustrated. The welding system 10 includes a welding helmet 12, a welding system 14, a welding gun 16 and a work piece 18. The work piece 18 generally defines a welding work area 20 where the welding gun 16 may be used to form a weld. The welding system 14 may be used for a variety of welding operations, including without limitation, welding, brazing, soldering, coating, hardfacing and cutting. While a welding gun 16 is illustrated, the welding system 14 may be used with a variety of implements, such as a laser, a waterjet, flame or arc generating torch or other system used in performing a welding operation.

The welding system 14 includes welding equipment 41 for generating a welding current and voltage, a welding control system 43 for controlling the welding current and voltage, and a monitoring system 45 for monitoring the welding current and voltage. The welding control system may also control a wire feeder at a desired wire feed speed for the welding operation. The monitoring system may also monitor a variety of other operating parameter, such as but not limited to, wire feed speed, amount of wire used/amount of wire remaining, any type of welding feedback desired by the operator and any other desired operating parameter.

The welding helmet 12 includes a main body 22 with a visual display system, generally indicated by the number 25, that includes display 24 connected to the main body 22. The display 24 may be a window including a welding lens, filter, a video monitor, such as an LCD display or LED array, or any other device suitable to allow a welder to see the welding work area 20. It must be understood that in such an example where the display 24 is a video monitor, video processing may be utilized to enhance the pictures of the welding operation. Further, recording devices may optionally be included to record and later playback welding operations for analysis and/or evaluation.

In various embodiments, the welding helmet 12 also includes a retinal tracking system 40. The retinal tracking system 40 is configured to identify a field of view of a user by tracking movement of the user's eyes. In some embodiments, the retinal tracking system detects a signal reflected by the retina of at least one of the welder's eyes and determines the welder's visual focus from the reflected signal.

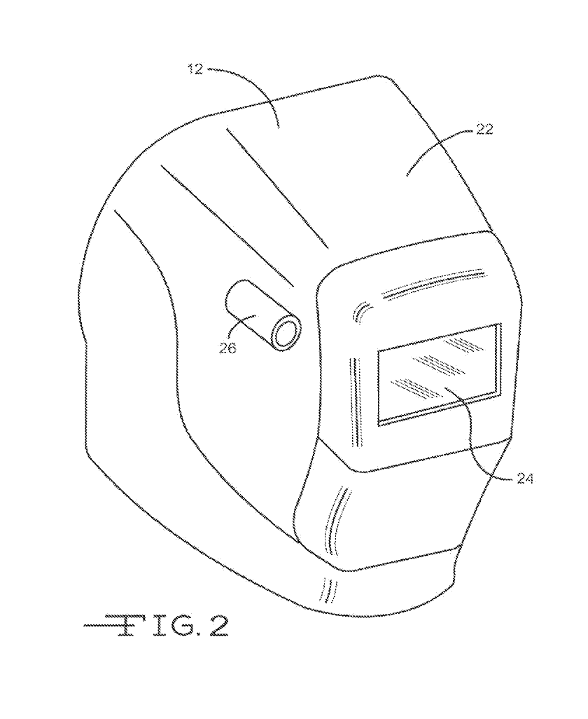

As best shown in FIG. 2, a welding helmet 12 may include a camera 26 mounted at or proximate to the point of view of the welder. In the example where the visual display 24 is a video monitor, the camera 26 may provide video pictures of the welding work area 20 to the display 24.

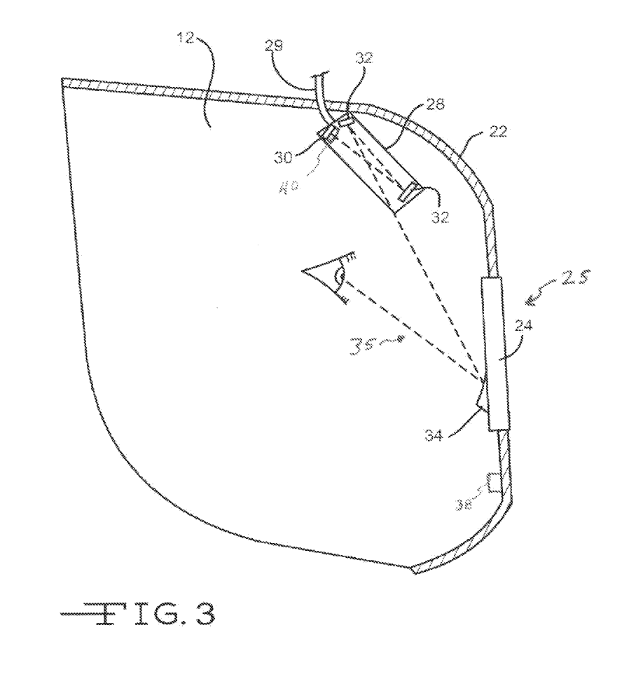

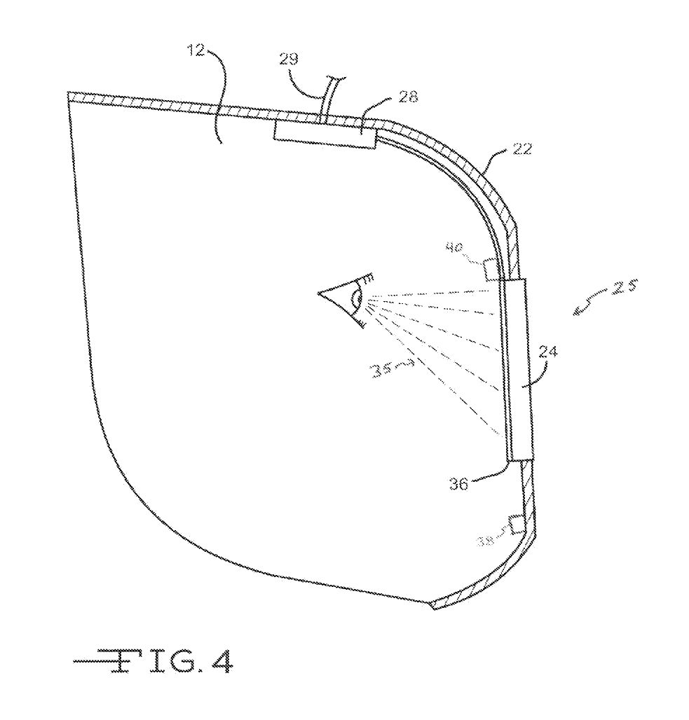

As shown in FIGS. 3 and 4 the visual display system 25 includes an information generating mechanism 28 in communication with the monitoring system of the welding system 14 and is capable of generating an image on the visual display 24. The generated image may be representative of information from the monitoring system relating to one or more monitored welding parameters, such as welding current and voltage. In some embodiments, the visual display system 25 provides the visual image such that the focus of the image is at a focus distance corresponding to an associated welding work area, e.g. outside of the main body 22 of the welding helmet 12. For example, the image may be symbolic, alpha-numeric, or any other device suitable to indicate the information. Thus, a welder may view an image representative of information about a welding operation without removing focus from the work area. In at least one embodiment the welder may focus on the work area and the image of information at the same time. In yet another embodiment, the visual display system may include a holographic projection system configured to generate the visual image of the welding parameters within the field of view of the welder.

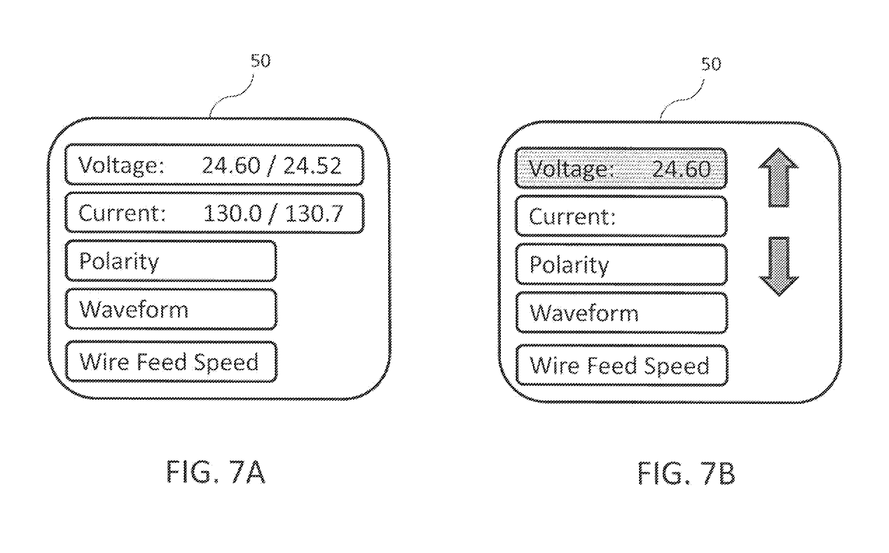

It must be understood that a variety of information and welding parameters may be displayed by the visual display system. For example, the displayed information may include information related to the welding current and voltage, such as welding current feedback, welding voltage feedback, control settings of the welding equipment, statistical information of the welding process, benchmarks or limits including capacity representations, alerts including material shortage or low flow, a representation of an intended or desired weld, etc. An example of one display is depicted in FIGS. 7A and 7B. This example is not limiting as other information and other configurations of the information may be used depending on the welding process, user preferences, and aesthetic considerations.

Further, in one embodiment, the camera 26 is used to calibrate the depth of the image relative to the welding work area 20. In another embodiment, positions sensors on the welding gun may be used to calibrate the depth of the image. In particular applications it is highly desirable to carefully align the image and the welding work are such that the information represented in the image is easy for the welder to access and such that the information in the image is readily accepted by the welder.

In the example where the visual display 24 is a video monitor, information generating mechanism 28 may include an image representative of information from the monitoring system based upon the monitored parameter, such as welding current and voltage, in video pictures of the welding work area 20 shown on the display 24.

As indicated at 29, the information generating mechanism 28 may be in wired or wireless communication with other devices as desired.

In FIG. 3, the information generating mechanism 28 is a projector 28. The projector may, for example, include an internal LCD display or LED array 30 along with a number of associated mirrors 32 to reflect the image generated to the visual display 24. The reflected image gives the image the appearance of depth relative to the visual display 24 and thus puts the image at a focus range with an associated welding work area that is outside of the main body 22 of the welding helmet 12 and optionally at the same focal distance as the associated welding work area 20. Optionally, a reflective surface 34 may be placed upon a portion of the visual display 24 in order to achieve a desired amount of reflection or reflection angle. In one embodiment, teleprompter type technology may be utilized to place the image upon the display 24 or surface 34. Additionally, it must be understood that one embodiment includes the use of an LCD display or other similar display within the helmet to generate the image which is then sent along an optical path, such as by reflection or fiber optics or any other suitable device to place the image on display 24 or surface 34.

In FIG. 4, the information generating mechanism 28 includes a screen, film, or sheet 36 integrated into the visual display 24. The sheet 36 may be a semi-transparent LCD film, electro-optic film, or any other suitable medium for the information generating mechanism 28 to produce an image generated in the visual display 24. In one application, the information generating mechanism 28 may project a stereogram on the welding lens such that a welder's eyes will separately view the images to create the perception of depth and thus focus the image at a focus range with the associated welding work area 20 and outside of the main body 22 of the welding helmet 12.

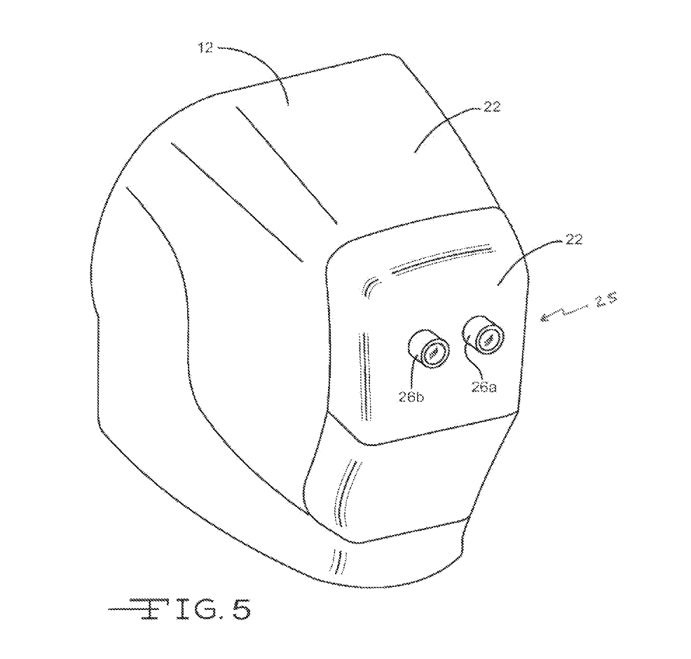

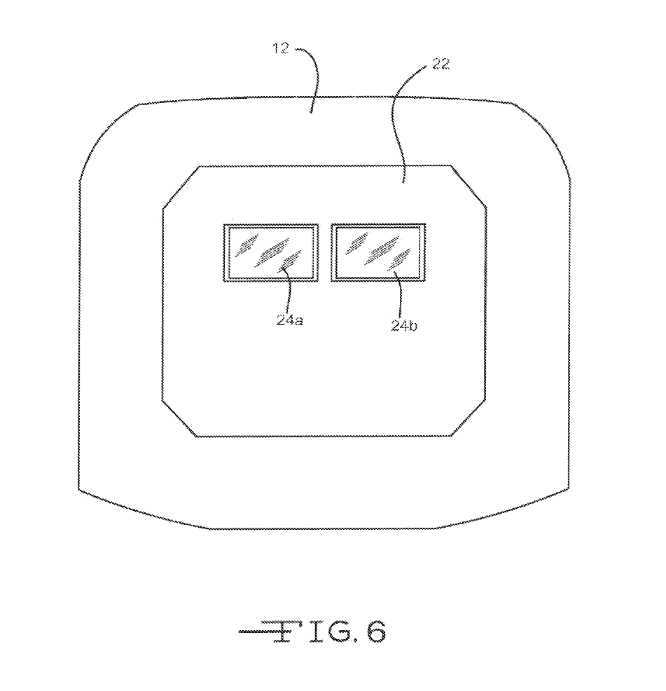

There is shown in FIG. 5 a welding helmet 12 including binocular cameras 26a and 26b. As shown in FIG. 6, these cameras 26a and 26b correspond to binocular viewing screens 24a and 24b. An information generating mechanism may produce an image to be generated in either of the viewing screens 24a or 24b or both. In one embodiment, the cameras 26a and 26b are placed in alignment with the screens 24a and 24b except on opposite sides of the main body 22, thus giving the welder the view directly in front of them. Additionally, in the embodiment with binocular cameras 26a and 26b and binocular viewing screens 24a and 24b the perception of depth of filed is produced.

In any case, the image may be an overlay of text or graphics or video feedback. Additionally, it is contemplated that in at least one embodiment the system described above may be used in a remote welding situation, including but not limited to robotic welding or underwater welding.

In each of the foregoing embodiments, the visual display system generates a visual image of the welding parameters. To improve the usability of the system, it may be desired to position the welding parameters within the field of view of the welder. The retinal tracking system 40 of the welding helmet is provided to identify the welder's field of view. As previously discussed, the retinal tracking system 40 may identify movement of the welder's eyes, such as by detecting a signal reflected from the at least one of the welder's retinas.

As shown in FIG. 2, the retinal tracking system 40 is positioned within the projector 28, co-located with LED array 30. In this configuration, the same mirrors 32 used to project the visual image of the welding parameters are also used to reflect the signal to/from the welder's retina. By monitoring changes in the reflected signal, the retinal tracking system 40 determines where a user is looking. The projector 28 may then shift the location of the projected visual image of the welding parameters so that they remain within the field of view 35 of the welder. In this manner, the visual display system 25 positions the visual image of the welding parameters within the field of view of the welder that was identified by the retinal track system.

In another embodiment, illustrated in FIG. 3, the retinal tracking system 40 is positioned in front of the welder adjacent the display 24. In this configuration, the retinal tracking system 40 has a direct line of sight to at least one of the welder's eyes enabling the movement of the eye to be identified and tracked. In yet other embodiments, the retinal tracking system may be provided on a monocle or spectacles (not shown) worn by the welder that interface with the welding helmet.

In addition to tracking movement of the welder's eyes to determine a field of view, the retinal tracking system 40 may also be configured to identify blinking or discrete eye movements which may be used to control one or more welding parameters.

Referring now to FIGS. 7A-B, an exemplary display/menu 50 of welding parameters is illustrated, such as may be projected by the visual display system. The display/menu 50 may include any of the welding parameters or related information discussed above, such as welding current and voltage, welding voltage feedback, control settings of the welding equipment, statistical information of the welding process, benchmarks or limits including capacity representations, alerts including material shortage or low flow, a representation of an intended or desired weld, etc.

Solely for purposes of illustrate, the display/menu 50 is illustrated as including welding voltage, welding current, polarity, waveform, and wire feed speed. For certain parameters, such as welding current and voltage, the display/menu identifies the parameter and provides the current value associated with that parameter. In some embodiments, the display/menu may include one or both of the current setting for the parameter, and the current monitored value of that parameter. In such embodiments, deviations from the current setting may be easily identified during a welding operation. As shown in FIG. 7A, the welding voltage is set to 24.60 volts, and the monitored value is 24.52 volts. Similarly, the welding current is set to 130.0 amps, and the monitored value is 130.7 amps.

The display/menu 50 provides a variety of functions for the welding system. As explained above, the display/menu operates to display the welding parameters and inform the welder about the current state of the welding process. In addition, the display/menu 50, in combination with the retinal tracking system 40, functions as a menu for modifying the welding parameters to control the welding process. In an embodiment, the retinal tracking system 40 is configured to receive welding parameter input from the welder based on tracking the movement of the welder's eyes. By way of illustration, the retinal tracking system 40 may be configured to identify specific eye movements or blink patterns, which correspond to menu selection commands. A double blink of the welder's left eye may cause the system to select and/or cycle through the displayed welding parameters. Once the desired parameter is selected (such as the welding voltage), a predetermined movement or blink pattern may be used to adjust that parameter to change the welding operation. After the desired adjustment has been completed, the system communicates the welding parameter input to the welding control system thereby controlling the welding process. In this manner, the welding current and voltage, as well as other welding parameters may be adjusted in an entirely hands-free operation, while the welder maintains visual awareness of the associated welding work area. As shown in FIG. 7B, the welder has selected the welding “voltage” parameter, making that parameter adjustable. The display/menu displays the current setting of 24.60 volts, and provides up and down arrows which may be used to adjust the setting.

In yet other embodiments, the welding helmet 12 includes a microphone 38. As illustrated in FIGS. 2-3, the microphone 38 may be positioned within the welding helmet generally oppose the position of the welder's mouth in order to receive audible commands from the welder. The microphone provides an additional control mechanism, which in combination with the retinal tracking system and visual display system may be used to adjust welding parameters to control at least one of the welding current and voltage. For example, while the retinal tracking system 40 may be used to navigate the display/menu 50, audio commands received through the microphone may be used to select items from the display/menu 50 or adjust parameters. By allowing for a limited vocabulary of commands, such as SELECT, SET, UP, DOWN, etc., a combination of eye-control and voice-control may be used. This combination of eye-control and voice-control may be sufficient for a welder to control all the applicable welding parameters which may be adjusted during a given welding operation in a hands-free manner, thereby significantly increasing the efficiency of the welding process to expand the hands-free capability of the system.

In another embodiment, the welding system receives a preset welding current and voltage for a desired welding operation. The present welding current and voltage are displayed in the welding helmet by the visual display system as previously discussed. In response to activating a trigger to begin a welding process, the visual display system automatically switches to display the actual welding current and voltage. In this manner, a welder is provided with real-time information on the status of the welding operation and can monitor for deviations from the desired welding current and voltage. In other embodiments, both the preset and actual current and voltage are simultaneously displayed by the visual display system.

In yet another embodiment, the visual display indicator is configured to display status indicators associated with one or more welding parameters. A welding procedure may define an acceptable range for various welding parameters, such as current and voltage and wire feed speed. During a welding operation, the actual value of the welding parameter may be compared to the acceptable range for that parameter defined by the welding procedure. So long as the welding parameter is within the acceptable range the visual display system may display the status indicator as green, as a plus sign, or other commonly understood indicator of a correct operating condition. If the welding parameter deviates from the acceptable range defined by the welding procedure, the status indicator may be displayed as red, as a negative sign, or other commonly understood indicator of an incorrect operating condition. In some embodiments, the status indicator may be the color of the displayed welding parameter value, while in other embodiments the status indicator may be a separate display. In yet other embodiments, a warning range may be defined for the welding parameters. A warning range may be defined by the welding procedure, or may be defined by the welding system based on percentage of the acceptable range. In such embodiments, the status indicator may be displayed as yellow or another commonly understood indicator of a caution condition, that may warrant attention from the operator so that the welding process may be corrected before deviating beyond the acceptable range for the welding parameter.

While principles and modes of operation have been explained and illustrated with regard to particular embodiments, it must be understood, however, that this may be practiced otherwise than as specifically explained and illustrated without departing from its spirit or scope.

What is claimed is:

1. A welding system comprising:
welding equipment for generating a welding current and voltage,
a welding control system for controlling the welding current and voltage,
a monitoring system for monitoring a welding parameter, and
a welding helmet including:
a main body,
a retinal tracking system configured to identify a field of view of a user by tracking movement of the user's eyes, and
a visual display system configured to generate a visual image of the welding parameter and further configured to position the visual image within the field of view of the user identified by the retinal tracking system.

2. The welding system of claim 1, wherein the retinal tracking system is further configured to receive a welding parameter input from the user based on tracking movement of the user's eyes, and to communicate the welding parameter input to the welding control system to control at least one of the welding current and voltage.

3. The welding system of claim 2, wherein the visual display system is further configured to display a menu of welding parameters, and the retinal tracking system is further configured to select a menu item from the menu of controllable welding parameters based on tracking movement of the user's eyes.

4. The welding system of claim 2, wherein the retinal tracking system is further configured to select a welding parameter, and the welding helmet further comprises a microphone configured to receive audible commands from the user to adjust the welding parameter to control at least one of the welding current and voltage.

5. The welding system of claim 2, wherein the welding parameter input includes at least one or more of the welding current, the welding voltage, and a wire feed speed.

6. The welding system of claim 1, wherein the retinal tracking system includes a monocle positioned between one of a user's eyes and the visual display system.

7. The welding system of claim 1, wherein the visual display system is a window including a welding lens.

8. The welding system of claim 7, wherein the visual display system is capable of projecting a stereogram on the welding lens.

9. The welding system of claim 7, wherein the visual display system includes a series of mirrors for reflecting the visual image toward the welding lens.

10. The welding system of claim 7, wherein the welding helmet includes a reflective surface proximate the welding lens for reflecting the visual image toward the interior of the welding helmet.

11. The welding system of claim 1, wherein the visual display system comprises a holographic projection system.

12. The welding system of claim 1, wherein the visual display system comprises includes an LCD display.

13. The welding system of claim 1, wherein the visual display system comprises includes an LED array.

14. The welding system of claim 1, wherein the visual display system includes at least one video monitor for displaying a picture of an associated welding work area.

15. The welding system of claim 14, wherein the welding helmet further includes at least one camera connected to the main body for providing the picture of the associated welding work area.

16. The welding system of claim 14, wherein the visual display system is configured to include the visual image of the welding parameter in the picture of the associated welding work area.

17. The welding system of claim 1, wherein the visual image includes alpha-numeric characters.

18. The welding system of claim 1, wherein the focus of the visual image is at the same focal distance as the associated welding work area.

19. The welding system of claim 1, wherein the visual display system is further configured to generate one or more status indicators each associated with a monitored welding parameter, wherein each status indicator indicates a relationship between the monitored welding parameter and an acceptable range for the monitored welding parameter defined by a welding procedure.
